(19) United States
(12) Patent Application Publication
(10) Pub. No.: US 2017/0287649 A1
(43) Pub. Date: Oct. 5, 2017

(54) PEROVSKITE SOLAR CELL HAVING HIGH HEAT RESISTANCE

(71) Applicant: KOREA RESEARCH INSTITUTE OF CHEMICAL TECHNOLOGY, Daejeon (KR)

(72) Inventors: Jang Won SEO, Seoul (KR); Jun Hong NOH, Daejeon (KR); Sang Il SEOK, Daejeon (KR); Nam Joong JEON, Gwangju (KR); Young Chan KIM, Daejeon (KR)

(21) Appl. No.: 15/478,618

(22) Filed: Apr. 4, 2017

(30) Foreign Application Priority Data
Apr. 5, 2016 (KR) 10-2016-0041448

(51) Int. Cl. H01G 9/20; H01L 51/00

(52) U.S. Cl. CPC H01G 9/2009; H01L 51/0078; H01L 51/0077; H01L 51/424

(57) ABSTRACT

Provided is a perovskite solar cell having remarkably excellent heat resistance, durability, and photoelectric conversion efficiency by employing a phthalocyanine derivative as a hole transport material.

### CROSS-REFERENCE TO RELATED APPLICATIONS

This application claims priority under 35 U.S.C. §119 to Korean Patent Application No. 10-2016-0041448, filed on Apr. 5, 2016, in the Korean Intellectual Property Office, the disclosure of which is incorporated herein by reference in its entirety.

### TECHNICAL FIELD

The following disclosure relates to a perovskite solar cell, and more particularly, to a perovskite solar cell including a hole transport layer which includes a phthalocyanine derivative in which heat transfer does not occur even in a wide temperature range to have excellent heat resistance, as a hole transport material.

### BACKGROUND

A perovskite solar cell, specifically, a lead halide perovskite solar cell, currently has an efficiency of 21% due to a photoactive layer of perovskite materials having excellent properties, as a result of a number of developments over many years.

The perovskite solar cell is the most commercialized among next generation solar cells including dye sensitized solar cells and organic solar cells, and thus, a full-scale study on stability and large area is required.

In particular, studies on deterioration mechanism of light, humidity, and heat among durability factors have been conducted, but a study on device heat stability is still in the early stage. As to heat stability of CH₃NH₃(=MA)PbI₃ and (NH₂)₂CH(=FA)PbI₃ which have been mainly studied as photoactive layers, FAPbI₃ exhibits a more improved property at a higher temperature of 85° C. or higher.

However, device stability has a drastic reduction in efficiency at a high temperature of 85° C. or higher, which is related to heat transfer at a high temperature of a widely used Spiro-OMeTAD single molecule itself.

Therefore, a heat-resistant hole transport material having no heat transfer even at a high temperature is required to be introduced as a means for solving the problem, and thus, various heat-resistant hole transport materials have been studied.

For example, a copper phthalocyanine derivative has been applied as a hole injection layer of an organic light emitting device, and is widely applied as a photoactive layer donor in an organic single molecular solar cell.

Recently, a zinc phthalocyanine derivative has been disclosed as a hole transport layer of a perovskite solar cell device.

However, a satisfactory photoelectric conversion efficiency has not been obtained yet.

### RELATED ART DOCUMENT

(Non-Patent Document) Dalton Trans, 2015, 44, 10847

### SUMMARY

An embodiment of the present invention is directed to providing a perovskite solar cell including a phthalocyanine derivative having excellent heat resistance as a hole transport material to have remarkably excellent properties, in particular, high stability and excellent photoelectric conversion efficiency as compared to conventional perovskite solar cells.

In one general aspect, a perovskite solar cell includes:

a phthalocyanine derivative represented by Chemical Formula 1 below:

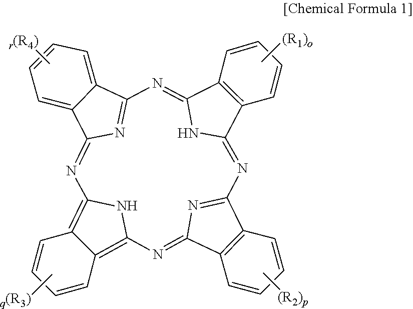

in Chemical Formula 1,

R₁ to R₄ are each independently (C1-C10)alkyl or (C1-C10)alkoxy, and

o, p, q and r are each independently 0 or an integer of 1 to 4, and when o, p, q and r are 2 or more, R₁ to R₄ may be the same as each other or different from each other.

Preferably, R₁ to R₄ in Chemical Formula 1 according to an exemplary embodiment of the present invention may be each independently (C1-C8)alkyl or (C1-C8)alkoxy.

Preferably, the Chemical Formula 1 according to an exemplary embodiment of the present invention may be represented by Chemical Formula 2 below:

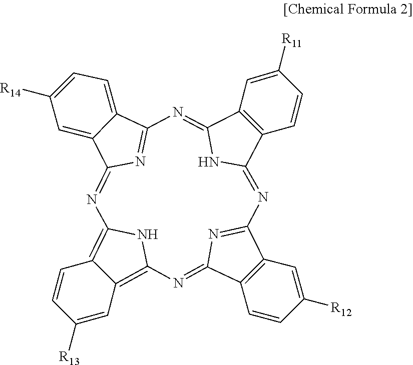

in Chemical Formula 2,

R₁₁ to R₁₄ are each independently (C1-C5)alkyl.

Preferably, R₁₁ to R₁₄ in Chemical Formula 2 according to an exemplary embodiment of the present invention may be tert-butyl or octyloxy.

Preferably, the phthalocyanine derivative according to an exemplary embodiment of the present invention may be used as a hole transport material of the perovskite solar cell, and the perovskite solar cell according to an exemplary embodiment of the present invention may include a first electrode, an electron transport layer formed on the first electrode, a light absorption layer including a compound having a perovskite structure and formed on the electron transport layer, a hole transport layer formed on the light absorption layer and including the phthalocyanine derivative represented by Chemical Formula 1, and a second electrode formed on the hole transport layer.

The hole transport layer according to an exemplary embodiment of the present invention may be formed by a solution process using the hole transport material including the phthalocyanine derivative represented by Chemical Formula 1.

The file of this patent contains at least one drawing executed in color. Copies of this patent with color drawing(s) will be provided by the Office upon request and payment of the necessary fee.

### BRIEF DESCRIPTION OF THE DRAWINGS

FIG. 1 is a graph showing photoelectric conversion efficiency depending on a temperature of perovskite solar cells according to Example 1 and Comparative Examples 1 and 2 of the present invention.

FIG. 2 is a graph showing durability of the perovskite solar cells according to Example 1 and Comparative Examples 1 and 2 of the present invention.

FIG. 3 is a graph showing current density of the perovskite solar cells according to Example 1 and Comparative Example 2 of the present invention.

### DETAILED DESCRIPTION OF EMBODIMENTS

The present invention provides a perovskite solar cell having excellent photoelectric conversion efficiency, durability, and stability. The perovskite solar cell of the present invention includes a phthalocyanine derivative represented by Chemical Formula 1 below:

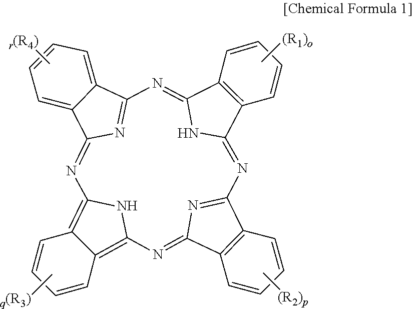

in Chemical Formula 1,

R₁ to R₄ are each independently (C1-C10)alkyl or (C1-C10)alkoxy, and

o, p, q and r are each independently 0 or an integer of 1 to 4, and when o, p, q and r are 2 or more, R₁ to R₄ may be the same as each other or different from each other.

The perovskite solar cell of the present invention may have significantly excellent stability and photoelectric conversion efficiency at a high temperature by employing the phthalocyanine derivative represented by Chemical Formula 1 in which heat transfer even does not occur in a wide temperature range to have excellent heat resistance, a hole transport ability is excellent, and at the same time, an oxidation stability is also excellent, as the hole transport material.

A perovskite solar cell employing a conventional spiro-OMeTAD [2,2′,7,7′-tetrakis (N,N-di-p-methoxyphenylamine)-9,9′-spirobifluorine] single molecule has remarkably deteriorated heat durability in which an efficiency is reduced at a high temperature of 85° C. or higher. This is because the Spiro-OMeTAD has a low heat transfer temperature (125° C.), causing damages such as pinholes, etc., in the thin film, thereby reducing durability and stability. The perovskite solar cell employing the phthalocyanine derivative represented by Chemical Formula 1 of the present invention overcomes these disadvantages, such that a crystal structure phase is not changed even at a high temperature, thereby securing stability.

Further, the perovskite solar cell employing the phthalocyanine derivative represented by Chemical Formula 1 of the present invention has remarkably high photoelectric conversion efficiency as compared to perovskite solar cells employing phthalocyanine in which an organic ligand is coordinated with a metal.

That is, the perovskite solar cell of the present invention has high durability and heat resistance to have remarkably improved photoelectric conversion efficiency while having excellent stability as compared to the perovskite solar cell employing phthalocyanine in which the organic ligand is coordinated with the metal, by employing a phthalocyanine derivative in which an organic ligand is not coordinated with a metal as a hole transport material rather than employing the phthalocyanine in which the organic ligand is coordinated with the metal.

In addition, the phthalocyanine derivative represented by Chemical Formula 1 has high solubility to a solvent, and thus, the hole transport layer including the phthalocyanine derivative is able to form a thin film by a solution process. Thus, the perovskite solar cell of the present invention is very economical and easily applied to commercial fields.

Preferably, in Chemical Formula 1 according to an exemplary embodiment of the present invention, R₁ to R₄ may be each independently C1-C8 alkyl or C1-C8 alkoxy, and more preferably, C1-C5 alkyl or C1-C5 alkoxy.

In view of high temperature stability and an increase in photoelectric conversion efficiency of the perovskite solar cell, the Chemical Formula 1 according to an exemplary embodiment of the present invention may be preferably represented by Chemical Formula 2 below:

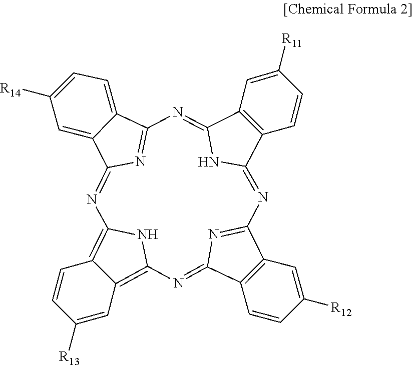

in Chemical Formula 2,

R₁₁ to R₁₄ are each independently C1-C5 alkyl.

In view of high temperature stability, photoelectric conversion efficiency, and solubility of the perovskite solar cell, R₁₁ to R₁₄ in Chemical Formula 2 according to an exemplary embodiment of the present invention may be preferably each independently tert-butyl or octyloxy (—OCH₂(CH₂)₆CH₃)), and more preferably, may be the same as each other as tert-butyl or octyloxy (—OCH₂(CH₂)₆CH₃)).

┌Alkyl┘, ┌alkoxy┘, and other substituents including ┌alkyl┘ part of the present invention include all linear or branched types, and 1 to 10 carbon atoms, preferably, 1 to 8 carbon atoms, and more preferably, 1 to 5 carbon atoms are included in the phthalocyanine derivative.

1 to 30 carbon atoms, and preferably, 1 to 25 carbon atoms are included in other parts except for the phthalocyanine derivative.

In addition, ┌aryl┘ used herein, which is an organic radical derived from aromatic hydrocarbon by removal of one hydrogen, includes single or fused ring system properly including 4 to 7 ring atoms, preferably, 5 or 6 ring atoms in each ring, and may even include a plurality of aryls linked with a single bond. Specific examples of the aryl include phenyl, naphthyl, biphenyl, anthryl, indenyl, fluorenyl, and the like, but the present invention is not limited thereto.

┌cycloalkyl┘ used herein refers to a non-aromatic monocyclic or multicyclic ring system having 3 to 20 carbon atoms, wherein the monocyclic ring includes cyclopropyl, cyclobutyl, cyclopentyl, and cyclohexyl, but is not limited thereto. Examples of a multicyclic cycloalkyl group include perhydronaphthyl, perhydroindenyl, etc.; and examples of a bridged multicyclic cycloalkyl group include adamantyl and norbornyl, etc.

Preferably, the phthalocyanine derivative according to an exemplary embodiment of the present invention may interact with the hole transport layer and a light absorption layer including a compound having a perovskite structure of a perovskite solar cell, thereby being used as a buffer layer, but preferably, may be used as the hole transport material of the perovskite solar cell.

The perovskite solar cell according to an exemplary embodiment of the present invention may include a first electrode, an electron transport layer formed on the first electrode, a light absorption layer including a compound having a perovskite structure and formed on the electron transport layer, a hole transport layer formed on the light absorption layer and including the phthalocyanine derivative represented by Chemical Formula 1, and a second electrode formed on the hole transport layer.

Parts corresponding to respective components of the perovskite solar cell according to an exemplary embodiment of the present invention include description of the international patent No. PCT-KR2014-012727 except for the hole transport layer which necessarily includes the phthalocyanine derivative represented by Chemical Formula 1.

Specifically, the first electrode according to an exemplary embodiment of the present invention may be any conductive electrode as long as it is ohmic-bonded to the electron transport layer, and the second electrode may be any conductive electrode as long as it is ohmic-bonded to the hole transport layer.

In addition, the first electrode and the second electrode may be any material as long as they are materials commonly used as an electrode material of a front electrode or a rear electrode in a solar cell. As a non-limiting example, when the first electrode and the second electrode are an electrode material of the rear electrode, the first electrode and the second electrode may be one or more materials selected from gold, silver, platinum, palladium, copper, aluminum, carbon, cobalt sulfide, copper sulfide, nickel oxide, and a composite thereof. As a non-limiting example, when the first electrode and the second electrode are transparent electrodes, the first electrode and the second electrode may be an inorganic conductive electrode such as fluorine doped tin oxide (FTO), indium doped tin oxide (ITO), ZnO, carbon nanotube (CNT), graphene, and an organic conductive electrode such as PEDOT:PSS. When it is attempted to provide a transparent solar cell, the first electrode and the second electrode are preferably transparent electrodes, and when the first electrode and the second electrode are organic conductive electrodes, it is more preferred when it is attempted to provide a flexible solar cell or a transparent solar cell.

The first electrode may be formed using deposition or application on a rigid substrate or a flexible substrate. The deposition may be formed by physical vapor deposition or chemical vapor deposition, and may be formed by thermal evaporation. The application may be performed by applying a solution in which the electrode material is dissolved or a dispersion solution of the electrode material on the substrate, followed by drying, or by heat-treating the selectively dried film. However, the first electrode and the second electrode may be formed by using methods for forming the front electrode or the rear electrode in conventional solar cells.

The electron transport layer formed on the first electrode of the present invention may be an electron conductive organic layer or an electron conductive inorganic layer. The electron conductive organic material may be an organic material used as an n-type semiconductor in conventional organic solar cells. As a specific and non-limiting example, the electron conductive organic material may include fullerenes (C60, C70, C74, C76, C78, C82, C95), fullerene derivatives including PCBM ([6,6]-phenyl-C61butyric acid methyl ester)), C71-PCBM, C84-PCBM, PC₇₀BM ([6,6]-phenyl C70-butyric acid methyl ester), PBI (polybenzimidazole), PTCBI (3,4,9,10-perylenetetracarboxylic bisbenzimidazole), F4-TCNQ (tetra uorotetracyanoquinodimethane) or a mixture thereof. The electron conductive inorganic material may be an electron conductive metal oxide used for electron transfer in conventional quantum dot-based solar cells or dye-sensitized solar cells. As a specific example, the electron conductive metal oxide may be an n-type metal oxide semiconductor. As a non-limiting example, the n-type metal oxide semiconductor may be one or two or more materials selected from Ti oxide, Zn oxide, In oxide, Sn oxide, W oxide, Nb oxide, Mo oxide, Mg oxide, Ba oxide, Zr oxide, Sr oxide, Yr oxide, La oxide, V oxide, Al oxide, Y oxide, Sc oxide, Sm oxide, Ga oxide, and SrTi oxide, and may be a mixture thereof or a composite thereof.

The light absorption layer formed on the electron transport layer according to an exemplary embodiment of the perovskite solar cell of the present invention includes a compound having a perovskite structure, and the compound having a perovskite structure may be any compound included within the range recognized by a person skilled in the art of the present invention.

For example, the compound having a perovskite structure means a compound containing a monovalent organic cation, a divalent metal cation, and a halogen anion and having a perovskite structure.

As a specific example, the compound having a perovskite structure of the present invention may be one or two or more materials selected from perovskite compounds satisfying Chemical Formulas 11 to 12 below:

AMX₃ [Chemical Formula 11]

in Chemical Formula 11, A is a monovalent organic ammonium ion or Cs⁺, M is a divalent metal ion, and X is a halogen ion, and

A₂MX₄ [Chemical Formula 12]

in Chemical Formula 12, A is a monovalent organic ammonium ion or Cs⁺, M is a divalent metal ion, and X is a halogen ion.

Here, M is positioned at the center of a unit cell in the perovskite structure, X is positioned at the center of each surface of the unit cell to form an octahedron structure around M, and A may be positioned at each corner of the unit cell.

Specifically, the light absorption layer may be each independently one or two or more selected from compounds satisfying Chemical Formulas 13 to 16 below:

(R₁—NH₃⁺)MX₃ [Chemical Formula 13]

in Chemical Formula 13, R₁ is C1-C24 alkyl, C3-C20 cycloalkyl or C6-C20 aryl, M is one or two or more metal ions selected from Cu²⁺, Ni²⁺, Co²⁺, Fe²⁺, Mn²⁺, Cr²⁺, Pd²⁺, Cd²⁺, Ge²⁺, Sn²⁺, Pb²⁺, and Yb²⁺, and X is one or two or more halogen ions selected from Cl⁻, Br⁻, and I⁻,

(R₁—NH₃⁺)₂MX₄ [Chemical Formula 14]

in Chemical Formula 14, R₁ is C1-C24 alkyl, C3-C20 cycloalkyl or C6-C20 aryl, M is one or more metal ions selected from Cu²⁺, Ni²⁺, Co²⁺, Fe²⁺, Mn²⁺, Cr²⁺, Pd²⁺, Cd²⁺, Ge²⁺, Sn²⁺, Pb²⁺, and Yb²⁺, and X is one or two or more halogen ions selected from Cl⁻, Br⁻, and I⁻,

(R₂—C₃H₃N₂⁺—R₃)MX₃ [Chemical Formula 15]

in Chemical Formula 15, R₂ is C1-C24 alkyl, C3-C20 cycloalkyl or C6-C20 aryl, R₃ is hydrogen or C1-C24 alkyl, M is one or two or more metal ions selected from Cu₂₊, Ni²⁺, Co²⁺, Fe²⁺, Mn²⁺, Cr²⁺, Pd²⁺, Cd²⁺, Ge²⁺, Sn²⁺, Pb²⁺, and Yb²⁺, and X is one or two or more halogen ions selected from Cl⁻, Br⁻, and I⁻, and

(R₂—C₃H₃N₂⁺—R₃)₂MX₄ [Chemical Formula 16]

in Chemical Formula 16, R₂ is C1-C24 alkyl, C3-C20 cycloalkyl or C6-C20 aryl, R₃ is hydrogen or C1-C24 alkyl, M is one or two or more metal ions selected from Cu₂₊, Ni²⁺, Co²⁺, Fe²⁺, Mn²⁺, Cr²⁺, Pd²⁺, Cd²⁺, Ge²⁺, Sn²⁺, Pb²⁺, and Yb²⁺, and X is one or two or more halogen ions selected from Cl⁻, Br⁻, and I⁻.

For example, the compound having a perovskite structure may be AMX^{a}ₓX^{b}_{y} or A₂MX^{a}ₓX^{b}_{y} (x is a real number of 0<x<3, y is a real number of 0<y<3, x+y=3, and X^{a} and X^{b} are halogen ions which are different from each other).

As an example, in Chemical Formula 13 or Chemical Formula 14, R₁ may be C1-C24 alkyl, preferably C1-C7 alkyl, and more preferably, methyl. As a specific example, the compound having a perovskite structure may be one or two or more selected from CH₃NH₃PbIₓCl_{y} (x is a real number of 0≦x≦3, y is a real number of 0≦y≦3, and x+y=3), CH₃NH₃PbIₓBr_{y} (x is a real number of 0≦x≦3, y is a real number of 0≦y≦3, and x+y=3), CH₃NH₃PbClₓBr_{y} (x is a real number of 0≦x≦3, y is a real number of 0≦y≦3, and x+y=3), and CH₃NH₃PbIₓF_{y} (x is a real number of 0≦x≦3, y is a real number of 0≦y≦3, and x+y=3), and further, may be one or two or more selected from (CH₃NH₃)₂PbIₓCl_{y} (x is a real number of 0<x<4, y is a real number of 0≦y≦4, and x+y=4), CH₃NH₃PbIₓBr_{y} (x is a real number of 0≦x≦4, y is a real number of 0≦y≦4, and x+y=4), CH₃NH₃PbClₓBr_{y} (x is a real number of 0≦x≦4, y is a real number of 0≦y≦4, and x+y=4), and CH₃NH₃PbIₓF_{y} (x is a real number of 0<x<4, y is a real number of 0≦y≦4, and x+y=4).

As an example, in Chemical Formula 15 or Chemical Formula 16, R₂ may be C1-C24 alkyl, R₃ may be hydrogen or C1-C24 alkyl, preferably R₂ may be C1-C7 alkyl, R₃ may be hydrogen or C1-C7 alkyl, and more preferably, R₂ may be methyl, and R₃ may be hydrogen.

The compound having a perovskite structure according to an exemplary embodiment of the present invention may preferably be represented by Chemical Formula 17 below:

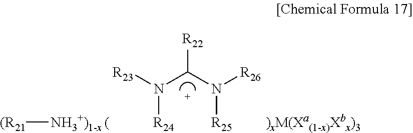

in Chemical Formula 17, R₂₁ is C1-C24 alkyl group, C3-C20 cycloalkyl group or C6-C20 aryl group, and R₂₂ to R₂₆ are each independently hydrogen, C1-C24 alkyl group, C3-C20 cycloalkyl group, or C6-C20 aryl group, M is a divalent metal ion, X^{a} is an iodine ion, X^{b} is a bromine ion, and x is a real number of 0.1≦x≦0.3.

Preferably, the light absorption layer according to an exemplary embodiment of the present invention may be a compound having a perovskite structure and containing lead.

The hole transport layer of the perovskite solar cell according to an exemplary embodiment of the present invention necessarily includes the phthalocyanine derivative represented by Chemical Formula 1 of the present invention.

Specifically, the hole transport layer of the present invention necessarily includes the phthalocyanine derivative represented by Chemical Formula 1 of the present invention as the hole transport material, and may include the phthalocyanine derivative alone, and may further include an organic hole transport material, an inorganic hole transport material, or a mixture thereof in addition to the phthalocyanine derivative represented by Chemical Formula 1. When the hole transport material is the inorganic hole transport material, the inorganic hole transport material may be an oxide semiconductor, a sulfide semiconductor, a halide semiconductor, or a mixture thereof which is a p-type semiconductor having hole conductivity. Examples of the oxide semiconductor may include NiO, CuO, CuAlO₂, CuGaO₂, etc., examples of the sulfide semiconductor may include PbS, and examples of the halide semiconductor may include PbI₂, etc. However, the present invention is not limited thereto.

When the hole transport material is an organic hole transport material, the hole transport material may include single molecular to polymeric organic hole transport materials (hole conductive organic materials). The organic hole transport material may be used as long as it is an organic hole transport material used in conventional inorganic semiconductor-based solar cells using inorganic semiconductor quantum dots as dye. A non-limiting example of the single molecular to low-molecular organic hole transport material may be one or two or more materials selected from pentacene, coumarin 6 [3-(2-benzothiazolyl)-7-(diethylamino)coumarin], ZnPC (zinc phthalocyanine), CuPC (copper phthalocyanine), TiOPC (titanium oxide phthalocyanine), spiro-OMeTAD (2,2′,7,7′-tetrakis(N,N-di-p-methoxyphenylamine)-9,9′-spirobifluorine), F16CuPC (copper(II) 1,2,3,4,8,9,10,11,15,16,17,18,22,23,24,25-hexadecafluoro-29H,31H-phthalocyanine), SubPc (boron subphthalocyanine chloride), and N3(cis-di(thiocyanato)-bis(2,2′-bipyridyl-4,4′-dicarboxylic acid)-ruthenium(II)).

The hole transport layer according to an exemplary embodiment of the present invention may be formed by a solution process using the hole transport material including the phthalocyanine derivative represented by Chemical Formula 1. The solution process to be performed according to an exemplary embodiment of the present invention may include, for example, screen printing, spin coating, bar coating, gravure coating, blade coating, and roll coating, etc. However, the present invention is not limited thereto.

Hereinafter, the present invention is described in detail with reference to specific Examples of the present invention, but is not intended to limit the scope of the claims of the present invention.

### Example 1

Manufacture of Porous TiO₂ Thin Film Substrate

A glass substrate coated with fluorine-containing tin oxide (FTO: F-doped SnO₂, 8 ohms/cm₂, Pilkington, hereinafter referred to as a FTO substrate (first electrode)) was cut into a size of 25×25 mm, and ends thereof were etched to partially remove the FTO.

A 50 nm thick TiO₂ dense film as a metal oxide thin film was prepared on the cut and partially etched FTO substrate by a spray pyrolysis method. The spray pyrolysis was performed by using a solution of TAA (titanium acetylacetonate):EtOH (1:9 v/v %), and the thickness was controlled by repeating a method of spraying the solution on the FTO substrate placed on a hot plate maintained at 450° C. for 3 seconds, followed by stopping of the spraying for 10 seconds.

An ethyl cellulose solution in which 10 wt % of ethyl cellulose was dissolved in ethyl alcohol, was added to TiO₂ powder having an average particle size (diameter) of 50 nm (prepared by hydrothermal treatment of an aqueous solution dissolved with titanium peroxocomplex of 1 wt % based on TiO₂ at 250° C. for 12 hours), wherein the ethyl cellulose solution was added in an amount of 5 ml per 1 g of TiO₂ powder, and terpinol (5 g per 1 g of TiO₂ powder) was added and mixed. Then, ethyl alcohol was removed by distillation under reduced pressure, thereby preparing a TiO₂ paste.

Ethanol was added to the prepared TiO₂ paste to prepare a TiO₂ slurry for spin coating. The TiO₂ thin film of the FTO substrate was coated with the TiO₂ slurry for spin coating by a spin coating method, and heat-treated at 500° C. for 60 minutes. Then, the heat-treated substrate was immersed in a 30 mM TiCl₄ aqueous solution at 60° and left for 30 minutes. Next, the substrate was washed with deionized water and ethanol, dried, and then, heat-treated at 500° C. for 30 minutes again to prepare a porous TiO₂ thin film (porous electron transport layer).

Preparation of Light Absorption Layer Solution

In a 250 mL two-neck round flask, 30 mL of hydriodic acid (HI) (57% in water, Aldrich) and 27.86 mL of formamidine acetate (FAAc, Aldrich) were reacted at 0° C. for 2 hours. The reaction mixture was distilled under reduced pressure at 50° C. for 1 hour. The obtained precipitate was dissolved in ethanol, recrystallized using ethyl ether, and dried at 60° C. for 24 hours to prepare NH₂CH═NH₂I(FAI).

The obtained NH₂CH═NH₂I (FAI), methylammonium iodide (CH₃NH₃I, MAI), and PbI₂ were mixed and dissolved in a mixed solution of γ-butyrolactone and dimethylsulfoxide (volume ratio 8:2), thereby preparing a light absorption layer solution so as to satisfy a composition of (FA_{0.85}MA_{0.15})Pb(I_{0.85}Br_{0.15})₃. The light absorption layer solution had a concentration of 0.96 M based on (FA_{0.85}MA_{0.15})Pb(I_{0.85}Br_{0.15})₃.

Manufacture of Perovskite Light Absorption Layer

The above-prepared light absorption layer solution ((FA_{0.85}MA_{0.815})Pb(I_{0.85}Br_{0.15})₃ solution) was coated on the above-manufactured porous TiO₂ thin film substrate (mp-TiO2/bl-TiO2/FTO) at 1000 rpm for 90 seconds, coated again at 5000 rpm for 30 seconds, and dried at 100° C. for 10 minutes, thereby manufacturing a light absorption layer. Here, 1 mL of toluene was added dropwise to the substrate in the second spin coating step.

Preparation of Hole Transport Layer Solution for Forming Hole Transport Layer

In order to form a hole transport layer, 0.01 g of the phthalocyanine derivative (Sigma-Aldrich) represented by compound 1 below as a hole transport material was dissolved in toluene to prepare a hole transport layer solution having a concentration of 10 mM. 10 μl of Li-bis (trifluoromethanesulfonyl) imide (Li-TFSI)/acetonitrile (170 mg/1 ml) and 5 μl TBP (4-tert-butylpyridine) as additives were added thereto, thereby preparing the hole transport layer solution:

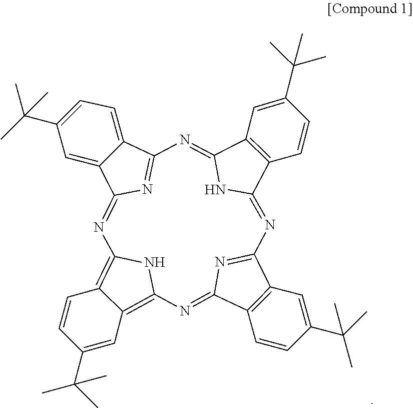 [SMILES: CC(C)(C)c1ccc2c(c1)-c1nc-2nc2[nH]c(nc3nc(nc4[nH]c(n1)c1ccc(C(C)(C)C)cc41)-c1ccc(C(C)(C)C)cc1-3)c1ccc(C(C)(C)C)cc21]

Manufacture of Perovskite Solar Cell

The above-prepared hole transport layer solution was subjected to spin coating at 3000 rpm for 30 seconds on a composite layer in which the above-manufactured light absorption structure was formed in the above-manufactured porous electrode, thereby forming a hole transport layer.

Next, Au was vacuum deposited on an upper portion of the hole transport layer by a thermal evaporator (5×10⁻⁶ torr or lower) to form an Au electrode (second electrode) having a thickness of 70 nm, and thus, Au/phthalocyanine/(FA_{0.5}MA_{0.15})Pb(I_{0.85}Br_{0.15})₃ (or represented by (FAPbI₃)_{0.85}(MAPbBr₃)_{0.15})/mp-TiO2/bl-TiO2/FTO solar cell was manufactured.

The electrode had an active area of 0.16 cm².

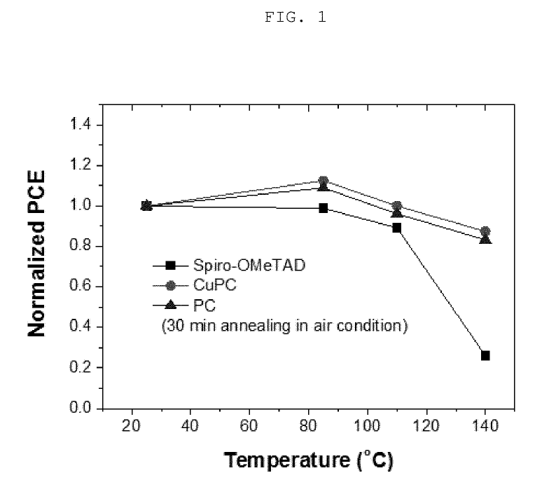

Properties of the manufactured solar cell were shown in FIGS. 1 to 3.

### Comparative Example 1

A perovskite solar cell was manufactured in the same manner as in Example 1 except that Spiro-OMeTAD [2,2′,7,7′-tetrakis (N,N-di-p-methoxyphenylamine)-9,9′-spirobi fluorine] was used instead of the phthalocyanine derivative, and properties of the manufactured solar cell were shown in FIGS. 1 to 3.

### Comparative Example 2

A perovskite solar cell was manufactured in the same manner as in Example 1 except that a compound 2 below (Sigma-Aldrich) was used instead of the phthalocyanine derivative, and properties of the manufactured solar cell were shown in FIGS. 1 to 3:

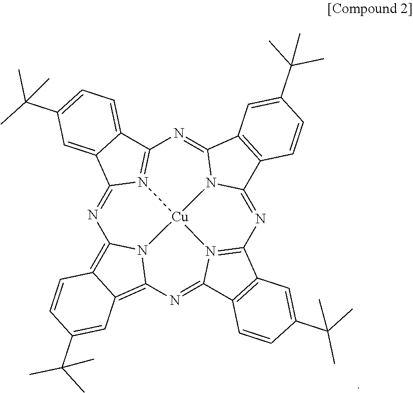

FIG. 1 shows photoelectric conversion efficiency depending on a temperature of the perovskite solar cells according to Example 1 (referenced by PC) and Comparative Example 1 (referenced by Spiro-OMeTAD) and Comparative Example 2 (referenced by CuPC). As shown in FIG. 1, the photoelectric conversion efficiency of the perovskite solar cells of Example 1 and Comparative Example 2 of the present invention were maintained even at a high temperature. On the contrary, the photoelectric conversion efficiency of the perovskite solar cell employing the Spiro-OMeTAD [2,2′,7,7′-tetrakis (N,N-di-p-methoxyphenylamine)-9,9′-spirobi fluorine] single molecule of Comparative Example 1 was rapidly deteriorated at 110° C.

Accordingly, it could be appreciated that the perovskite solar cell of the present invention had high heat resistance.

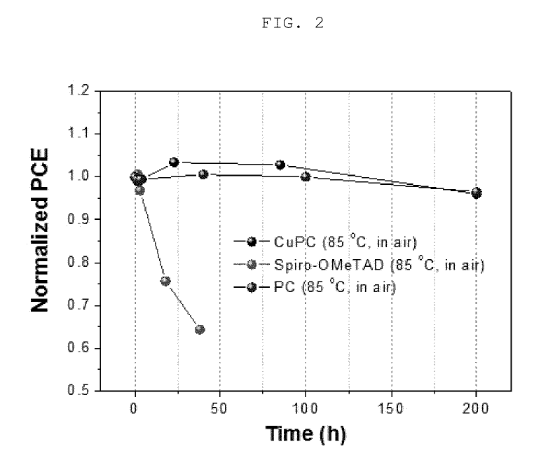

Further, in order to measure the durability of the perovskite solar cells of Example 1 and Comparative Examples 1 and 2 of the present invention, the photoelectric conversion efficiency was measured after the perovskite solar cells were left for 200 hours at a temperature of 85° C. and an average relative humidity of 25 to 30%, and this measurement was performed two times in total. That is, the durability of the perovskite solar cells of Example 1 (referenced by PC) and Comparative Example 1 (referenced by Spiro-OMeTAD) to 2 (referenced by CuPC) was shown in FIG. 2 with the photoelectric conversion efficiency after a lapse of a predetermined time compared to the initial value. As shown in FIG. 2, it could be appreciated that the photoelectric conversion efficiency of the perovskite solar cells of Example 1 and Comparative Example 2 of the present invention was maintained as 96% or more as compared to the initial value without a significant change over time, but the photoelectric conversion efficiency of that of Comparative Example 1 was remarkably reduced.

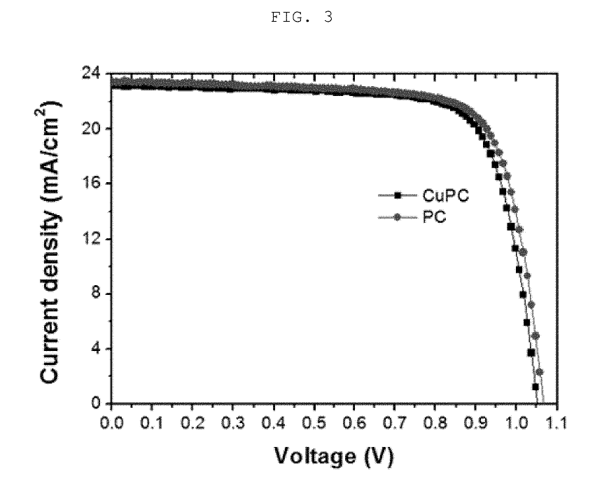

Further, FIG. 3 and Table 1 below show the photoelectric conversion efficiency of the perovskite solar cells of Example 1 (referenced by PC) and Comparative Example 2 (referenced by CuPC) of the present invention.

TABLE 1

| | J_{sc} | Voc | FF | PCE |
|---|---|---|---|---|
| | (mA/cm²) | (V) | (%) | (%) |
| Example 1 | 23.5 | 1.06 | 77.5 | 19.3 |
| Comparative | 23.2 | 1.05 | 75.5 | 18.3 |
| Example 2 | | | | |

As shown in Table 1 and FIG. 3, it could be appreciated that the perovskite solar cell of Example 1 employing the phthalocyanine derivative which was not coordinated with the metal of the present invention had remarkably increased photoelectric conversion efficiency as compared to the perovskite solar cell of Comparative Example 2 employing the phthalocyanine derivative which was coordinated with the metal. That is, it could be appreciated that the perovskite solar cell of Example 1 of the present invention had a higher photoelectric conversion efficiency by about 5.4% or more, which is not a level that is able to be easily derived by a person skilled in the art, as compared to the perovskite solar cell of Comparative Example 2.

The perovskite solar cell of the present invention may have significantly high durability, heat resistance, and storage stability by employing the phthalocyanine derivative in which heat transfer does not occur even in a wide temperature range to have excellent heat resistance, as the hole transport material.

In addition, the perovskite solar cell of the present invention may have a high photoelectric conversion efficiency at a high temperature, that is, even at a temperature of 85° C. or higher, and further, may maintain the high photoelectric conversion efficiency even at a temperature of 85° C. or higher over a long time by employing the phthalocyanine derivative as the hole transport material.

Further, the perovskite solar cell of the present invention is very economic since it is able to form the hole transport layer by an inexpensive solution process rather than an expensive deposition process.

What is claimed is:

1. A perovskite solar cell comprising:
a phthalocyanine derivative represented by Chemical Formula 1 below: 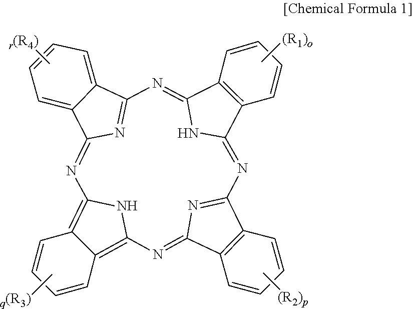
in Chemical Formula 1,
R₁ to R₄ are each independently (C1-C10)alkyl or (C1-C10)alkoxy, and
o, p, q and r are each independently 0 or an integer of 1 to 4, and when o, p, q and r are 2 or more, R₁ to R₄ are the same as each other or different from each other.

2. The perovskite solar cell of claim 1, wherein R₁ to R₄ are each independently (C1-C8)alkyl or (C1-C8)alkoxy.

3. The perovskite solar cell of claim 1, wherein the Chemical Formula 1 is represented by Chemical Formula 2 below: 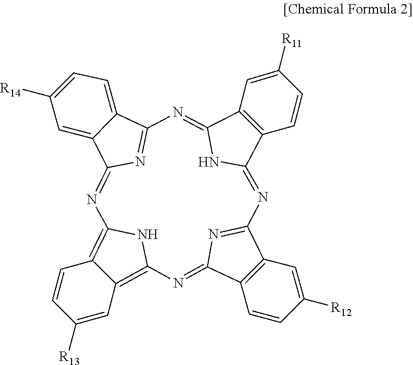
in Chemical Formula 2,
R₁₁ to R₁₄ are each independently (C1-C5)alkyl.

4. The perovskite solar cell of claim 1, wherein R₁ to R₄ are each independently tert-butyl or octyloxy.

5. The perovskite solar cell of claim 1, wherein the phthalocyanine derivative is used as a hole transport material of the perovskite solar cell.

6. The perovskite solar cell of claim 1, wherein the perovskite solar cell includes a first electrode, an electron transport layer formed on the first electrode, a light absorption layer including a compound having a perovskite structure and formed on the electron transport layer, a hole transport layer formed on the light absorption layer and including the phthalocyanine derivative represented by Chemical Formula 1, and a second electrode formed on the hole transport layer.

7. The perovskite solar cell of claim 6, wherein the hole transport layer is formed by solution casting with a hole transport material including the phthalocyanine derivative represented by Chemical Formula 1.
